(19) United States
(12) Patent Application Publication
(10) Pub. No.: US 2008/0228004 A1
(43) Pub. Date: Sep. 18, 2008

(54) Ligand Protection for Mercaptoacetyl Triglycine

(76) Inventors: Mart-Jan T. Blauwhoff, Overath (DE); Hechtor H. Knight, Alkmaar (NL); Friedhelm R. Stock, Schagen (NL); Volcmar Verkerk, Amsterdam (NL)

Correspondence Address: Jerad Seurer;Mallinckrodt, 675 McDonnell Boulevard, P.O.Box 5840, St. Louis, MO, 63134, US

(21) Appl. No.: 10/585,792

(22) PCT Filed: Feb. 11, 2005
(86) PCT No.: PCT/US2005/004349

§ 371 Date: Jul. 12, 2006

(30) Foreign Application Priority Data
Feb. 13, 2004 (EP) 04075448.3

(51) Int. Cl. C07C 233/47

(52) U.S. Cl. 564/153

(57) ABSTRACT

The present invention relates to a method for preparing mercaptoacetyl triglycine labeled with a radionuclide, comprising the steps of adding a radionuclide to a solution that comprises a mercaptoacetyl triglycine dimer of formula VI, a reducing agent and optionally a transfer ligand and heating the thus obtained solution. The invention relates to the mercaptoacetyl triglycine dimer and its use in the method, to a kit for performing the method and to the formulation obtained from the method.

The present invention relates to a method for preparing mercaptoacetyl triglycine labeled with a radionuclide. The invention further relates to a S-protected mercaptoacetyl triglycine compound and a kit for use in this method, and to a formulation comprising radiolabeled mercaptoacetyl triglycine.

Mercaptoacetyl triglycine (MAG3) labeled with Tc-99m is a diagnostic radiopharmaceutical. It is supplied as a lyophilized powder comprising betiatide (N-[N-[N-[(benzoylthio)acetyl]glycyl]glycyl]glycine) with suitable reducing agent and transfer ligand. After reconstitution with sterile sodium pertechnetate Tc-99m, the Tc-99m mertiatide (disodium[N-[N-[N-(mercaptoacetyl)glycyl]glycyl] glycinato-(2-)-N,N′,N″,S′]oxotechnetate(2-)) which is formed is suitable for intravenous administration.

Tc-99m mertiatide is a renal imaging agent for example for use in the diagnosis of congenital and acquired kidney abnormalities, such as renal failure, urinary tract obstruction, and calculi in adults and children. It is a diagnostic aid in providing information about renal function, split function, renal angiograms and renogram curves for whole kidney and renal cortex. It is furthermore used in functional studies of the kidney after transplantation in which repeated doses are administered.

During the preparation of the ^{99m}Tc-mercaptoacetyl triglycine (MAG3) complex at slightly acidic conditions (pH 5-6), the thiol is protected by a benzoyl group, which, in turn is removed during the 10 minutes' boiling step to allow coordination to the metal center. It might be more convenient not to have benzoic acid in the final preparation, due to its possible toxicity.

It is therefore the object of the invention to provide an alternative method for preparing a solution of mercaptoacetyl triglycine labeled with a radionuclide.

In the research that led to the present invention it was found to be possible to use the mercaptoacetyl triglycine itself as “protecting group”. Upon reconstitution, both the Tc-99m and the MAG3-dimer are reduced simultaneously to afford the desired product.

The invention thus relates to a method for preparing mercaptoacetyl triglycine labeled with a radionuclide, comprising the steps of adding a radionuclide to a solution that comprises a mercaptoacetyl triglycine dimer of formula VI

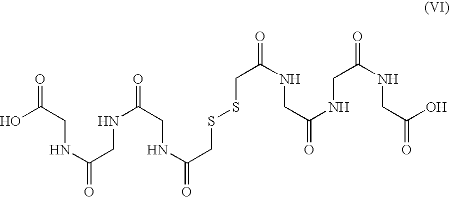

a reducing agent and optionally a transfer ligand and heating the thus obtained solution.

The radiolabeled mercaptoacetyl triglycine is obtained in solution. In a preferred embodiment the solution that comprises the mercaptoacetyl triglycine dimer, the reducing agent and the optional transfer ligand is obtained by reconstitution from a lyophilisate.

The radionuclide for use in the method of the invention can be any radionuclide that can be bound to the mertiatide complex, and is suitable for radiodiagnostic or radiotherapeutic purposes and is preferably technetium-99m Technetium-99m is the preferred radionuclide as Tc-99m is the most desirable radioactive label for diagnostic applications. It emits low energy (140 KeV) radiation, which is well-suited for use in combination with standard radiation-measuring instrumentation. In addition, it is inexpensive and its half-life is only about 6 hours, which together with its lack of emission of beta particles during its decay results in very low radiation dose per millicurie. These properties make Tc-99m ideal as a tool in nuclear medicine. Suitably the technetium is added as ^{99M} Tc-pertechnetate.

The reducing agent is a stannous salt, preferably stannous (II) chloride. Other examples are Fe(III)-, Sb(III)-, Mo(III)- and W(III)-salts. The transfer ligand is suitably selected from sodium tartrate, glycine, citrate, malonate, gluconate, malate, lactate, pyrophosphate, glucoheptonate. Of these tartrate is preferred.

It was found that Tc-99m complex is only formed when the solution is heated to 80-120° C., preferably to 100° C. during 5-60 minutes, preferably during about 10 minutes.

The method of the invention avoids the use of benzoyl protecting group.

The invention further relates to the dimer of mercaptoacetyl triglycine according to formula VI and its use in the method.

The invention also provides a kit for the preparation of a radiolabeled mercaptoacetyl triglycine complex, comprising a dimer of mercaptoacetyl triglycine according to formula VI, a reducing agent and optionally a transfer ligand.

In a preferred embodiment the kit comprises in lyophilized form:

0.05 mg MAG3-dimer
0.14 mg tin(II) chloride.2aq
17.2 mg disodium tartrate.2aq.

The kit is in lyophilised form as this leads to a better stability and longer shelf-life.

In a further aspect thereof, the invention relates to a formulation of mercaptoacetyl triglycine labeled with a radionuclide, which is obtainable by the method. Since the mercaptoacetyl triglycine is not protected with a benzoyl potecting group, the formulation that is obtained does not contain benzoic acid as a part of the injectable, while being formulated at physiologically acceptable pH (no need to neutralize prior injection into the patient).

The formulation may further comprise the usual constituents. For example, a suitable reducing agent is needed. The actual formulation can contain stannous chloride, while disodium tartrate can function as stabilizer of the Tc(V) oxidation state and transfers ligand simultaneously. The resulting product has the same or higher radiochemical purity and stability and the same or longer shelf-life.

The invention will be illustrated in the Examples that follow and in which reference is made to the following figures:

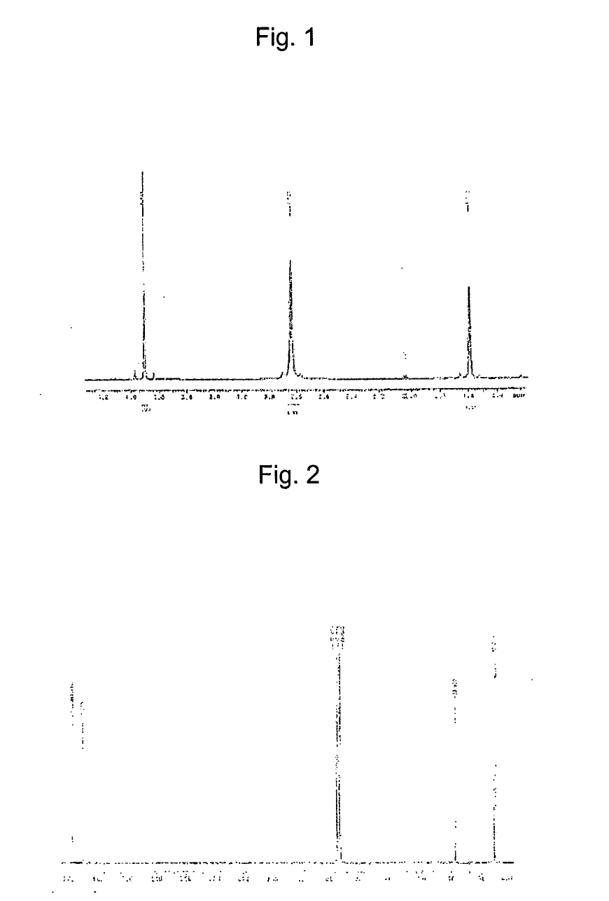

FIG. 1 shows the ¹H-NMR spectrum of the MAG3 dimer precursor.

FIG. 2 shows the ¹³C-NMR spectrum of the MAG3 dimer precursor.

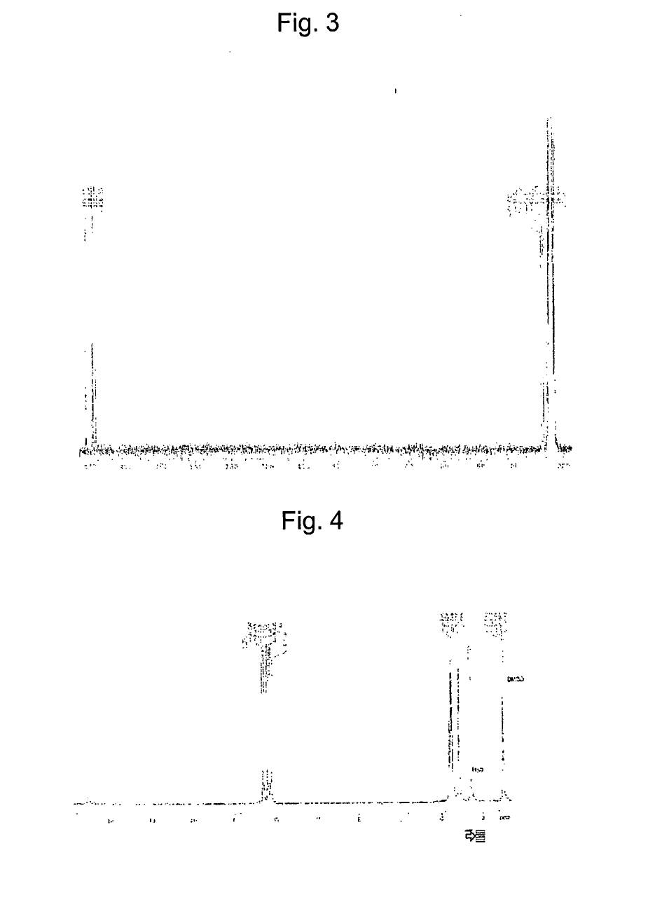

FIG. 3 shows the ¹³C-NMR spectrum of the MAG3 dimer.

FIG. 4 shows the ¹H-NMR spectrum of the MAG3 dimer.

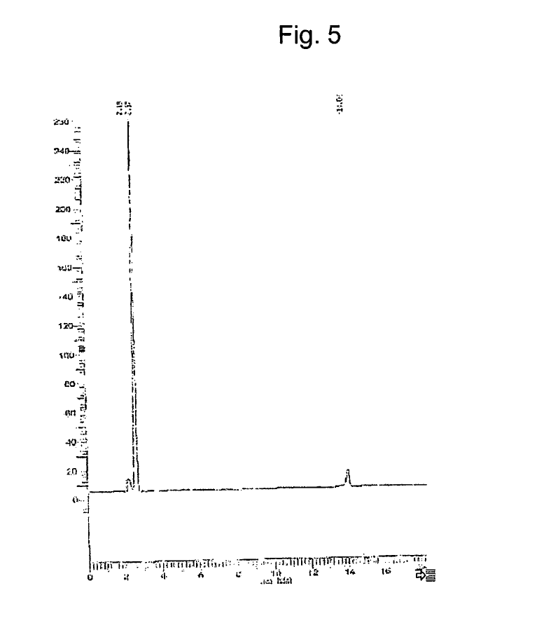

FIG. 5 shows the HPLC profile of the MAG3 dimer.

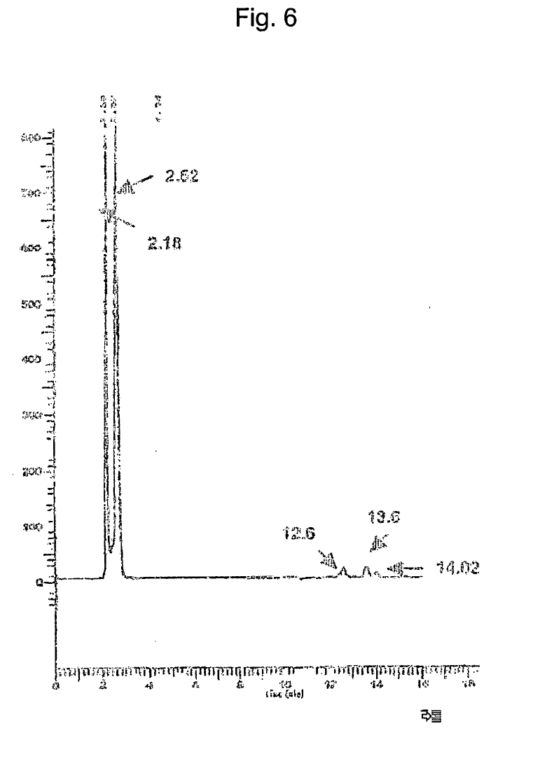

FIG. 6 shows the HPLC chromatogram obtained by co-injecting the MAG3 dimer and its monomer.

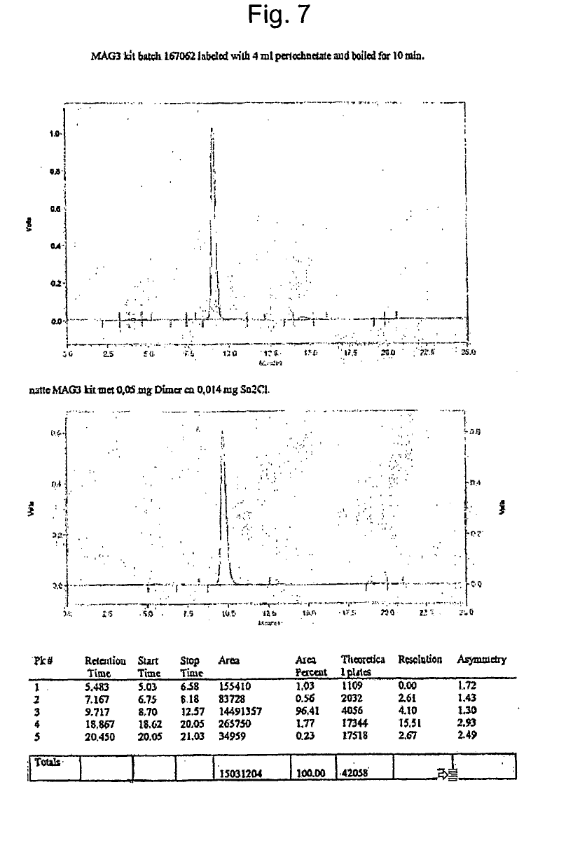

FIG. 7 shows two examples of HPLC chromatograms obtained (1) for the official product Technescan MAG3 after labeling and (2) for the labeled “wet” formulation containing the dimer as active ingredient.

### EXAMPLES

### Example 1

### Synthesis and Characterization of Mercaptoacetyl Triglycine-Dimer (MAG3)₂ (VI)

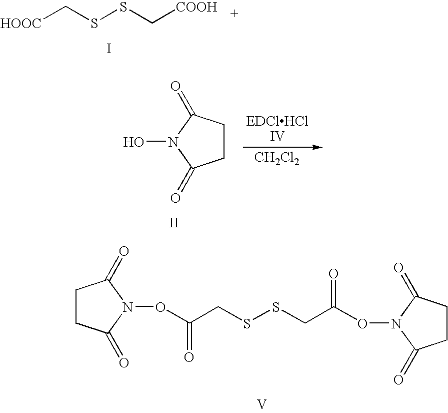

### Synthetic Route

### 1. Synthesis and Characterization of the Activated Ester V

A solution of 2.0 g (10.96 mmol) dithioglycolic acid (I) in 30 ml dry dichloro-methane is cooled to 0° C. in an ice bath. N-Hydroxysuccinimide (II) (2.78 g, 24.2 mmol) and 4.64 g (24.2 mmol) of 1-(3-dimethylaminopropyl)-3-ethyl-carbodiimide hydrochloride (EDCl.HCl, IV) are added and the reaction mixture is stirred at 0° C., under nitrogen for 30 minutes, later at room temperature for one hour. The solvent is evaporated and the solid residue is washed three times with water. The activated ester is vacuum dried and purified, first by column chromatography (on silica gel with 10% methanol in dichloromethane as eluent) and finally by recrystallization from ethyl acetate. The purified product (2 g, 5.4 mmol) is obtained in 49% yield

### Elemental Analysis for C₁₂H₁₂N₂O₈S₂:

| | Calculated | Found |
|---|---|---|
| C | 3831 | 3830 |
| N | 746 | 744 |
| H | 385 | 321 |
| S | 1716 | 1704 |

FIG. 1 shows the ¹H-NMR spectrum. The corresponding chemical shifts are as follows:

### 1. 3.90 ppm (s, 4H, S—CH₂)

### 2. 2.84 ppm (s, 8H, CH₂)

3. 1.58 ppm, (s, H₂O from the CDCl₃)

### CDCl₃, 7.24 ppm

FIG. 2 shows the ¹³C-NMR spectrum. The corresponding chemical shifts are as follows:

### 1. 168.76; 165.10 ppm (CO)

### 2. 38.70; 25.58 ppm (CH₂)

### CDCl₃, 77.0 ppm

### 2. Synthesis and Characterization of the MAG3 Dimer ((MAG3)₂)

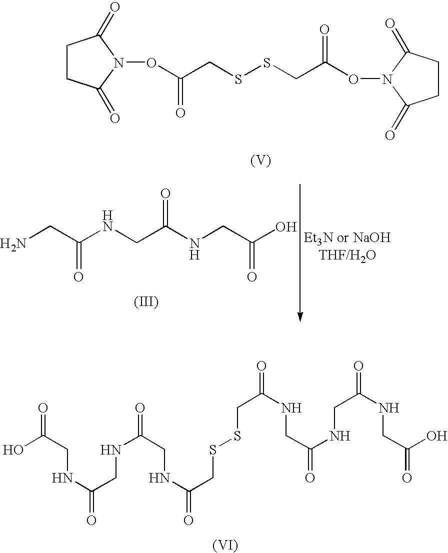

A solution of 200 mg (0.53 mmol) of the activated ester (V) in 10 ml of THF, is cooled down to 0° C. on an ice bath. A suspension of triglycine (VI) (201 mg, 1.06 mmol) in 1 ml water and 1 ml of sodium hydroxide 1 N is slowly added to the solution above. The reaction mixture is stirred at room temperature for 2 hours to yield a yellow solution, which is vacuum dried to remove the THF. The remaining aqueous solution is acidified with HCl 2N until precipitation starts (∀ 3 ml). The precipitate formed is collected by filtration and washed several times with water, until the pH of the filtrate is 5. The white solid obtained is vacuum dried to afford 195 mg (0.37 mmol) of the final product. The yield is 69%. This step can also be performed with triethylamine (NEt₃) instead of sodium hydroxide (NaOH). The yield is then a bit lower, 59%.

### 3. Purification of the (MAG3)₂

The crude product is dissolved in 6 ml of a 3.5% solution of sodium hydrogen carbonate (NaHCO₃). Hydrochloric acid 2N is added until a white precipitate appears. The solid is filtered off and washed several times with water until the pH of the filtrate is 5. The product is vacuum dried overnight.

### 4. Characterization of (MAG3)₂

### 4.1 Elemental Analysis for C₁₆H₂₄N₆O₁₀S₂:

| | Calculated | Found |
|---|---|---|
| C | 3664 | 3673 |
| N | 1602 | 1599 |
| H | 461 | 478 |
| S | 1223 | 1213 |

### 4.2 Ellman's Test

A calibration curve was made from a standard solution of Cysteine in Phosphate buffer 0.1 M, pH 8. The samples were dissolved in phosphate buffer 0.1 M, pH 8 to give a concentration of 2 mM. The absorbance was measured at 412 nm and the concentration of free thiols (SH) was calculated from the calibration curve.

Two batches of (MAG3)2 were analyzed by spectrophotometry.

| Batch | Absorb. | [—SH]mM | % |
|---|---|---|---|
| 1 | 8 | 119 | 3 |
| 2 | 14 | 206 | 5 |

### 4.3 NMR Spectroscopy

### 4.3.1 ¹³C-NMR Spectrum

FIG. 3 shows the ¹³C-NMR spectrum. The chemical shifts were as follows:

C1, 171.16 ppm; C2, 169.14 ppm; C3, 169.01 ppm; C7, 168.38 ppm; C4, 42.36 ppm; C5, 41.88 ppm; C6, 41.76 ppm; CB, 40.60 ppm; DMSO, 39.5 ppm

### 4.3.2 ¹H-NMR

FIG. 4 shows the ¹H-NMR spectrum. The chemical shifts were as follows:

### 1. 12.53 ppm (1H, s, br, OH)

### 2. 8.31 ppm (1H, tr., J_{N-H}, 6 Hz, N—H); 8.19 ppm (1H, tr., J_{N-H},

### 6 Hz, N—H); 8.12 ppm (1H, tr., J_{N-H}, 6 Hz, N—H)

### 3. 3.78 ppm (2H, d., J_{H-H}, 6 Hz, CH₂); 3.74 ppm (4H, d., J_{H-H},

### 6 Hz, 2CH₂)

### 4. 3.56 ppm (2H, s, S—CH₂)

### DMSO, 2.49 ppm

### 4.4 HPLC Analysis

The following parameters were used:

| | |
|---|---|
| Column | Hypersil ODS 10 mm |
| Mobile phase | A: 0.1% TFA in water |
| | B: acetonitrile |
| Gradient | 0-5 min 100% A |
| | 5-10 min 0 to 10% B |
| | 10-20 min 10% B |
| Flow | 1 ml/min |
| Detection | UV, 254 nm |

The result is shown in FIG. 5.

The monomer, mercaptoacetyl triglycine, obtained by reducing the disulfide bond in the (MAG3)₂ was co-injected with the parent compound to give the chromatogram shown in FIG. 6.

### Example 2

### Formulation Experiments

In order not to alter the composition of the kit formulation with respect to the existing MAG3 kit, stannous chloride was used as reducing agent and sodium tartrate as transfer ligand. A formulation containing 0.5 mg of the MAG3 dimer, 17.1 mg sodium tartrate dihydrate and 0.047 mg Sn(II)Cl₂ afforded, after a 10 minutes' boiling step in the presence of ^{99m}TcO⁴⁻, between 60 and 70% of ^{99m}Tc-MAG3.

The standard Technescan MAG3 formulation (reference) contains:

1 mg benzoylmercaptoacetyl triglycine (Benzoyl MAG3)
0.04 mg Tin(II) chloride
16.9 mg disodium tartrate

The MAG3-dimer formulation of the invention contains for example:

0.05 mg MAG3-dimer
0.14 mg tin(II) chloride
17.2 mg disodium tartrate

FIG. 7 shows two examples of HPLC chromatograms obtained (1) for the official product Technescan MAG3 after labeling and (2) for the labeled “wet” formulation containing the dimer as active ingredient.

1. Method for preparing mercaptoacetyl triglycine labeled with a radionuclide, comprising:
adding a radionuclide to a solution that comprises a mercaptoacetyl triglycine dimer of formula VI and a reducing agent; and 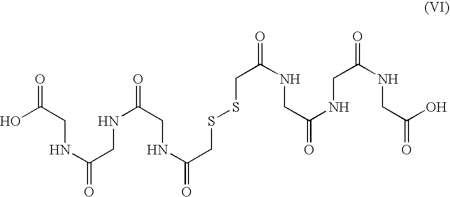
heating the solution at least after the adding.

2. Method as claimed in claim 1, wherein the solution that comprises the mercaptoacetyl triglycine dimer and the reducing agent is obtained by reconstitution from a lyophilisate.

3. Method as claimed in claim 1, wherein the radionuclide is technetium-99m.

4. Method as claimed in claim 3, wherein the adding comprises adding ^{99m}Tc-pertechnetate.

5. Method as claimed in claim 1-4, wherein the reducing agent is selected from stannous salts.

6. Method as claimed in claim 16, wherein the transfer ligand is selected from sodium tartrate, glycine, citrate, malonate, gluconate, malate, lactate, pyrophosphate, and glucoheptonate.

7. Method as claimed in claim 1, wherein the heating comprises heating the solution to 80-120° C.

8. Method as claimed in claim 1, wherein the heating occurs for a duration of 5-60 minutes.

9. Radiolabeled mercaptoacetyl triglycine prepared according to the method of claim 1.

10. Kit for the preparation of a radiolabeled mercaptoacetyl triglycine complex, comprising:
a dimer of mercaptoacetyl triglycine according to formula VI; and 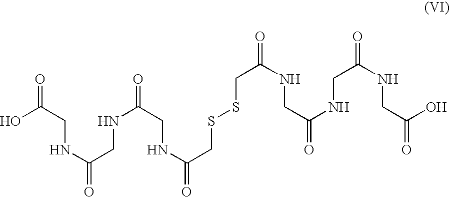
a reducing agent.

11. Kit as claimed in claim 10, wherein the reducing agent comprises a stannous salt.

12. Kit as claimed in claim 2, wherein the transfer ligand is selected from sodium tartrate, glycine, citrate, malonate, gluconate, malate, lactate, pyrophosphate, and glucoheptonate.

13. Kit as claimed in claim 10, comprising:
0.01-0.10 mg MAG3-dimer; and
0.05-0.25 mg tin(II) chloride.

14. Kit as claimed in claim 10, which is in lyophilised form.

15. Formulation of radiolabeled mercaptoacetyl triglycine prepared using the kit of claim 10.

16. Method as claimed in claim 1, wherein the solution comprises a transfer ligand.

17. Method as claimed in claim 1, wherein the reducing agent comprises stannous chloride.

18. Method as claimed in claim 1, wherein the heating comprises heating the solution to about 100° C.

19. Method as claimed in claim 1, wherein the heating occurs for a duration of about 10 minutes.

20. Kit as claimed in claim 10, further comprising:
a transfer ligand.

21. Kit as claimed in claim 10, wherein the reducing agent comprises stannous chloride.

22. Kit as claimed in claim 13, further comprising:
10-20 mg disodium tartrate.
